(19) United States
(12) Patent Application Publication
(10) Pub. No.: US 2025/0375748 A1
(43) Pub. Date: Dec. 11, 2025

(54) Fluid Treatment Device and Method

(71) Applicants: Julien Clerdy, Liège (BE); François Royen, Donceel (BE); David Thonon, Tilff (BE)

(72) Inventors: Julien Clerdy, Liège (BE); François Royen, Donceel (BE); David Thonon, Tilff (BE)

(73) Assignee: ELYSIA, Liège (BE)

(21) Appl. No.: 19/230,440

(22) Filed: Jun. 6, 2025

(30) Foreign Application Priority Data
Jun. 7, 2024 (BE) BE2024/5336

(51) Int. Cl. B01J 8/00

(52) U.S. Cl. CPC B01J 8/00; B01J 2219/004; B01J 2219/00522; B01J 2219/0068

(57) ABSTRACT

A fluid treatment device is disclosed. The device includes: a cassette including a pre-assembled tubing, the cassette having a flat surface for receiving the tubing, the tubing including at least one flexible tube able to be pinched; at least one pinch valve including a movable pin and a fixed stop for pinching the flexible tube in order to control a fluid flow rate in the flexible tube; wherein the pinch valve is mechanically coupled to the cassette such that the movable pin of the pinch valve describes a trajectory parallel to the flat surface of the cassette when the pinch valve is activated to pinch the flexible tube, and wherein the tubing may be easily, quickly, and cost-effectively installed and replaced. Further disclosed is a fluid treatment method allowing a tubing included in a fluid treatment device according to the disclosure to be easily, quickly, and cost-effectively installed and replaced.

### CROSS-REFERENCE TO RELATED APPLICATION

This application claims priority to Belgian Patent Application No. BE 2024/5336 filed Jun. 7, 2024, the disclosure of which is hereby incorporated by reference in its entirety.

### TECHNICAL FIELD

The embodiments of the present disclosure relate to the field of fluid treatment for the analysis and synthesis of chemical compounds, in particular the field of analysis and synthesis of radiopharmaceutical compounds, via fluidic and micro-fluidic treatment devices and methods allowing the manipulation and the precise control of fluid flow rates. Preferably, the disclosure concerns such devices configured for a small number of uses, or even for a single use.

### BACKGROUND

In the field of analysis and synthesis of radiopharmaceutical compounds, complex fluidic flow diagrams are generally created using a manifold. In the context of this document, the term “manifold” refers to a system for distributing and controlling the flow rate of one or more fluids, such as a set of connections and valves allowing various combinations of tube connections, where a fluid may be a liquid or a gas. A manifold may comprise the following elements: tubes to transport a fluid; joints to connect different tubes; valves to control, i.e. regulate or interrupt, a flow rate of a fluid; connectors to connect tubes to items of equipment. A manifold implements a “fluidic flow diagram”, i.e. a configuration of tubes, junctions, valves, connectors, etc. allowing a given method for treating one or more fluids to be carried out.

The use of conventional manifolds, where the fluid circulating through the manifold is in contact with manifold valves, may have a number of disadvantages. Firstly, it may be difficult to clean these valves, which may lead to risks of contamination of a fluid by another fluid previously circulating in the manifold. In addition, there may be residual fluid portions in the valves, which may lead to fluid losses, these lost fluids also being referred to as “dead volumes”. Finally, there may be problems of chemical stability because the fluids circulating in the manifold may react with the materials making up the valves and junctions. In the case of a small number of uses, or even a single use, of a conventional manifold, where the fluid circulating in the manifold is in contact with manifold valves, it may be necessary to dispose of these valves after use, which may pose several problems, such as a large volume of waste generated, or the significant cost of replacing the manifold. If the fluid circulating in the manifold contains a radioactive compound, generating a large volume of potentially radioactive waste may be problematic in that this potentially radioactive waste must undergo specific treatment, which may be costly.

In order to overcome these disadvantages, it is possible to separate the valves themselves from the tubes carrying the fluid, which has led to the creation of so-called “tube pinch” valves, where a valve controls a flow rate of a fluid in a flexible tube by pinching the tube, thus avoiding any direct contact between the valve itself and the fluid being transported, thus eliminating the problems of contamination and also of dead volumes at the level of the valves of a manifold.

There are at least two main categories of tube pinch valves, hereinafter also referred to simply as “pinch valves”. Firstly, pneumatic pinch valves, where the valve pinch mechanism is operated by means of compressed air. Then there are pinch solenoid valves, which comprise an electromagnet and a return spring, and use an electric current through the electromagnet to actuate the valve's pinching mechanism.

However, a number of problems remain with the use of pinch valves to control the flow rate of a fluid through one or more tubes or a network of tubes, hereinafter also referred to as “tubing”. This is because it may not be easy to separate the tubing from the valve pinch mechanism in order to install or replace the tubing, for example in the case of a tubing for a small number of uses or single use. In addition, in the case of a fluid treatment device comprising a tubing for a small number of uses or single use, it may be desirable to have pre-assembled tubing, which may then be more easily and quickly installed and replaced. In particular, for certain applications such as the synthesis of pharmaceutical or radiopharmaceutical compounds, it may be necessary to manipulate the tubing in an isolator that allows the tubing to be installed through gloves, such as a “glove box” type device. In this case, it may be difficult to handle the tubing and a tubing that is not pre-assembled may be difficult to install in a fluid treatment device. Finally, it may be desirable for such a device comprising a tubing for a small number of uses or single use to require few resources to manufacture, in order to limit the cost of manufacturing and/or replacing the tubing.

The document U.S. Pat. No. 5,901,745A describes a device comprising a series of pinch valves and their respective flexible tubes. One disadvantage of this device is that it does not allow the tubing to be installed or replaced quickly and easily. This means that the entire device has to be dismantled to install or replace the tubing, which may be very costly.

There is therefore a need for a fluid treatment device comprising a tubing that may be easily, quickly, and cost-effectively installed and replaced.

### SUMMARY

The present disclosure provides examples of a fluid treatment device comprising, for example, a tubing that may be easily, quickly, and cost-effectively installed and replaced.

In an embodiment of the present disclosure, the fluid treatment device comprises: a cassette comprising a pre-assembled tubing, wherein the cassette has a substantially flat surface for receiving the tubing. The tubing comprises at least one flexible tube that may be pinched (or able to be pinched). The fluid treatment device also comprises at least one pinch valve comprising a movable pin (or lug) and a fixed stop for pinching the flexible tube in order to control a fluid flow rate in the flexible tube. The at least one pinch valve is mechanically coupled to the cassette such that the movable pin of the at least one pinch valve describes a trajectory substantially parallel to the substantially flat surface of the cassette when the at least one pinch valve is activated to pinch the flexible tube.

For the purposes of the present disclosure, the term “tubing” refers to one or more tubes, and in some embodiments, a network of tubes. Such a network of tubes may comprise at least one branch. For the purposes of the present disclosure, a “cassette” refers to a body for supporting a tubing. Such a cassette is, for example, a case or cartridge that can be easily mounted or unmounted. Such a cassette has, for example, a flat shape. A tube pinch valve, also referred to simply as a “pinch valve” in the present disclosure, is used to control, i.e. regulate or interrupt, a flow rate of fluid in a flexible tube of a tubing by pinching the flexible tube.

The pinch valve provided in the fluid treatment devices disclosed herein is able to pinch the flexible tube between the movable pin and the fixed stop in order to control a flow rate of fluid circulating in the flexible tube. In other words, the flexible tube comprised in the tubing may be pinched by the pinch valve. More specifically, the flexible tube may be pinched between the movable pin and the fixed stop of the pinch valve.

The tubing of the fluid treatment devices disclosed herein may be easily, quickly, and cost-effectively installed and replaced. The cassette comprising the tubing is separate from the pinch valve containing the movable pin and the fixed stop. In this way, the cassette comprising the pre-assembled tubing may be mechanically coupled to (or mounted on) the pinch valve. In an embodiment, the cassette may be detachably coupled to the pinch valve. For example, the cassette may be coupled with (or decoupled from) the pinch valve without having to dismantle the pinch valve. In other words, the cassette comprising the pre-assembled tubing is ready to use (“plug and play”). In this way, the cassette may be handled, i.e. positioned and replaced (or simply removed), independently of the at least one pinch valve.

In addition, when the pinch valve is activated, the movable pin comprised in the pinch valve describes a trajectory substantially parallel to the substantially flat surface of the cassette. Furthermore, in the case where the fluid treatment device comprises several pinch valves, these valves are located on a plane essentially parallel to the essentially flat surface of the cassette. The trajectory of the movable pin of each pinch valve comprised in the fluid treatment device therefore does not follow a direction essentially perpendicular to the flat surface of the cassette, leaving this perpendicular direction free for coupling the tubing comprised in the cassette to each pinch valve.

In this way, the cassette comprising the tubing may be installed in a single movement along a trajectory whose direction is essentially perpendicular to the essentially flat surface of the cassette and oriented towards the at least one pinch valve comprised in the fluid treatment device, in order to couple the tubing with at least one pinch valve. The tubing comprised in the cassette may therefore be mechanically coupled with the assembly of the pinch valves in a single operation. In one or more embodiments, it is not necessary to adjust the tubing individually to each of the pinch valve or valves.

Similarly, the tubing comprised in the cassette may be mechanically decoupled from the assembly of the pinch valves in a single movement, following a trajectory whose direction is essentially perpendicular to the essentially flat surface of the cassette and oriented away from the at least one pinch valve. The tubing comprised in the cassette may therefore be mechanically decoupled from the assembly of the pinch valves in a single operation. In one or more embodiments, it is not necessary to individually decouple the tubing from each of the pinch valve or valves.

The tubing comprised in the fluid treatment device may therefore be installed and replaced quickly and easily. In addition, as the cost of placing or replacing the tubing may be proportional to the time taken by an operator to install or replace the tubing, the fact that these operations may be carried out quickly means that they may be carried out at a lower cost.

In an embodiment, the tubing is pre-assembled in the cassette. The fact that the tubing is pre-assembled in the cassette means that the tubing is assembled with the cassette before the cassette is coupled with the at least one pinch valve. The cassette does not comprise a movable pin or a fixed stop, the latter two elements being comprised in a pinch valve separate (or distinct) from the cassette. In other words, the cassette comprising the tubing is independent from the pinch valve comprising the movable pin and the fixed stop. The fact that the tubing is pre-assembled in the cassette means that the tubing may be installed and replaced quickly and easily on the device according to the disclosure.

Another advantage of having the tubing pre-assembled in the cassette is the ability to constrain or control one or more characteristic dimensions of the tubing, such as a length of tubing or a diameter of tubing.

In addition, the fact that the tubing is pre-assembled in the cassette means that an operator may install or replace the cassette in a single operation, i.e. quickly and easily. In fact, it is not necessary to connect the tubes of the tubing together when placing or replacing the cassette comprised in a device according to the disclosure. During such placement or replacement, it is therefore simply necessary to connect the tubes to the other fluid handling or analysis equipment that may be comprised in a fluid treatment apparatus comprising an embodiment of fluid treatment device. In addition, the fact that the tubing comprised in the cassette is pre-assembled allows to limit or reduce the risk of error when placing or replacing such tubing in the fluid treatment device by limiting or reducing the number of operations required for such placement or replacement. In addition, having the tubing pre-assembled in the cassette means that the tubing may be easily and quickly extracted from the fluid treatment device, with a reduced risk of leakage or loss of liquid, which is advantageous, particularly in the case of a radioactive liquid.

In addition, the fluid treatment device according to the disclosure is generic. This means that the pinch valve or valves comprised in the fluid treatment device may be coupled with different tubing configurations. In other words, the pinch valve or valves may be coupled with different cassettes, each cassette comprising a pre-assembled tubing in a specific configuration. This is made possible by the fact that the cassette comprises the tubing but does not comprise a pinch valve. In other words, the fact that the tubing is pre-assembled in the cassette means that the configuration of the tubing may be decoupled from the configuration of the pinch valves. This makes the fluid treatment device disclosed herein generic, i.e. capable of accommodating different tubing configurations where each tubing configuration corresponds to a different cassette. Thus, an operator wishing to implement a specific fluid treatment scheme (or fluidic flow diagram) on the fluid treatment device will be able to select a cassette comprising a pre-assembled tubing and configured in such a way as to be able to implement this specific fluidic flow diagram, and couple this cassette with the pinch valve or valves comprised in the fluid treatment device, thus allowing a rapid and simple implementation of this fluidic flow diagram.

Further, when a pinch valve is activated, no force is directly applied to the cassette by the movable pin comprised in the pinch valve. The stop against which the movable pin comprised in a pinch valve pinches a flexible tube to control a fluid flow rate in the flexible tube is not comprised in the cassette, but forms part of the pinch valve. As a result, the cassette does not need to be particularly rigid and may therefore be made from a less robust material, requiring fewer resources to manufacture. The cassette may therefore be manufactured at lower cost, and may therefore be installed and replaced at lower cost. Furthermore, as no force is directly applied to the cassette when a pinch valve is activated, the cassette does not have to be held firmly in place when it is mechanically coupled with the pinch valve or valves of the fluid treatment device. This facilitates the placement and the replacement of the cassette by limiting the number of operations required for such placement or replacement.

Advantageously, the cassette in some embodiments does not comprise any movable mechanical parts. This makes it easier to sterilize the cassette.

The inventors propose several possible embodiments of the disclosure comprising optional characteristics, where some of them may be combined or omitted.

According to one embodiment, the fluid treatment device further comprises a base comprising the at least one pinch valve, wherein the cassette is mechanically coupled with (or mounted on) the base such that the movable pin of the at least one pinch valve describes a trajectory essentially parallel to the essentially flat surface of the cassette when the at least one pinch valve is activated to pinch the flexible tube.

The tubing in this embodiment may also be installed and replaced easily, quickly and cost-effectively. Indeed, the cassette comprising the tubing is separate (or independent) from the base containing the at least one pinch valve. The cassette may be mechanically coupled to (or mounted on) the base comprising the pinch valve. For example, the cassette comprising the pre-assembled tubing may be simply placed on the base comprising the pinch valve. In an embodiment, the cassette may be removably coupled to the base. In this way, the cassette may be easily and quickly coupled with the base so as to mechanically couple the tubing comprised in the cassette with the at least one pinch valve comprised in the base.

In another embodiment, the cassette may be coupled to the base without having to dismantle the base or any part of the base. In other words, the cassette comprising the pre-assembled tubing is ready to use (“plug and play”). In this way, the cassette may be handled, i.e. installed and replaced (or simply removed), independently of the at least one pinch valve comprised in the base. In other words, the cassette may be coupled with or inserted into the base independently of the pinch valve, i.e. independently of the assembly and disassembly of the pinch valve. The cassette may also be decoupled or separated from the base independently of the pinch valve, i.e. independently of the assembly and disassembly of the pinch valve.

In an embodiment, the cassette is coupled to the base removably and independently of the at least one pinch valve. This makes it even easier to install and replace the tubing by making it easier to install and replace the cassette comprising the pre-assembled tubing.

According to another embodiment, the cassette also comprises a network of support channels for accommodating (or receiving) the pre-assembled tubing. The network of support channels comprises, for each pinch valve, a clearance slot allowing the movable pin and the fixed stop to be mounted around the flexible tube that may be pinched by the pinch valve. As the tubing may be partially or totally made up of flexible tubes, it is advantageous for the cassette to comprise a network of support channels to receive the tubing. Such a network of support channels allows to maintain the tubing in a desired predefined arrangement, thus facilitating the coupling of the tubing comprised in the cassette with the at least one pinch valve. Such a network of support channels may take the form of grooves cut into the thickness of the cassette and may comprise at least one branch. For example, the support channels may be in the form of grooves extending longitudinally along the tubing.

In an embodiment, a clearance slot is provided in the cassette at the level of each flexible tube section of the tubing configured to be pinched by a pinch valve. In the case of a pinch valve, this clearance slot accommodates the fixed stop and the movable pin which are positioned on either side of a section of flexible tube that may be pinched by the valve, thereby facilitating the coupling of the cassette with the at least one pinch valve comprised in the fluid treatment device. Such a clearance slot may be in the form of a hole, for example, an oval hole, made in the thickness of the cassette, such a hole may be, for example, a blind hole or a hole passing through the thickness of the cassette.

In an embodiment, the pre-assembled tubing is held in the network of support channels by clamping. As the tubing may be partially or totally made of flexible tubes, it is possible to provide a tubing whose outer diameter is adjusted to the width of the support channels so that the tubing may be held in the support channel network by clamping. This configuration limits the resources required to maintain the tubing in the network of support channels. This configuration also makes it easier to place the tubing into the cassette.

In general, other ways of holding the tubing in the cassette are possible. For example, the tubing may be held in or on the cassette by hooks, notches, or grooves into which all or part of the tubing may be inserted, or by other techniques.

In one embodiment, the cassette is essentially made of a thermoplastic polymer. As described above, when a pinch valve is activated, no force is directly applied to the cassette by the movable pin comprised in the pinch valve. As a result, the cassette does not need to be particularly rigid and may therefore be made from a less robust material, requiring fewer resources to manufacture. A thermoplastic polymer, such as polymethylmethacrylate (PMMA), polycarbonate (PC) or polypropylene (PP), is an example of such a low-strength material and offers the following advantages. A thermoplastic polymer may be inexpensive and easy to shape. A cassette essentially made of a thermoplastic polymer may thus be manufactured in large series, for example by molding, requiring little material and at a low unit manufacturing cost. Such a cassette may therefore be adapted for a small number of uses, or even a single use, for example in the case where such a cassette needs to be replaced frequently.

In one or more embodiments, the thermoplastic polymer of which the cassette is essentially made has any one or more of the following characteristics: the thermoplastic polymer is translucent, and preferably transparent, so that it is possible in particular to see the tubing comprised in the cassette, to check the assembly of the tubing, and to visualize the path of the fluid in the tubing; the thermoplastic polymer is non-porous; the thermoplastic polymer is sterilizable, so that the cassette may be reused, thus limiting the volume of waste generated when the tubing comprised in the device according to the disclosure is replaced.

In one embodiment, the pre-assembled tubing comprised in the cassette comprises at least one connector allowing the tubing to be coupled to at least one device capable of manipulating the fluid or measuring a physico-chemical property of the fluid. The following are examples of devices capable of handling a fluid, i.e. allowing one or more fluids to be supplied, received or moved through the tubing: a fluid sample flask, possibly sealed by a septum; a fluid reservoir; a syringe, possibly operated by a syringe pump, and possibly equipped with a needle, in order to take or inject a fluid sample; a pump, such as a peristaltic pump for example; a valve; a mixer; a filter; a heating device; a cooling device; or other devices. Other examples of physico-chemical properties of the fluid that may be measured include temperature, pH, radioactivity, the concentration in the fluid of a chemical or biochemical compound, such as its concentration in endotoxins, or other properties. Such physico-chemical properties may be measured, for example, by the following devices, to which the tubing may be coupled: a temperature may be measured by a thermometer; a pH may be measured by a pH meter; a radioactivity may be measured by a scanner of the “radio-TLC” type (for “thin layer chromatography”), or by a detector of the gamma counter type, or of the multi-channel counter type, or even a gas counter such as an ionization chamber; a concentration of endotoxins may be measured by an apparatus for analysis by spectrophotometry. The tubing may also be coupled to other measuring devices such as chromatography apparatus, such as gas chromatography apparatus or liquid chromatography apparatus, or other devices. Further, the following examples of connectors for coupling the tubing to any one or more of the aforementioned devices, or to other devices, may be cited: so-called “Luer” connectors, such as “Luer Slip” or “Luer-Lock”, designed to provide a leak-tight connection between a syringe and a needle, and also to provide a leak-tight connection between a tubing and a syringe, or between a tubing and a needle.

In an embodiment, the tubing of the fluid treatment device comprises, or in some embodiments, consists of, a network of essentially cylindrical tubes with an outer diameter of between 1 mm and 10 mm, for example between 2 mm and 3 mm, and in a certain embodiment equal to 2.4 mm. The outer diameter of the tubing is at least 1 mm to allow a sufficient flow rate of fluid through the tubing, and at most 10 mm to limit fluid losses, also referred to as “dead volumes”, i.e. the volume of fluid remaining in the tubing after the treatment of the fluid and which may not be easily extracted from the tubing and is therefore lost. In an embodiment, the outer diameter of the tubing is at least 2 mm to allow sufficient fluid flow rate through the tubing and also to make it easier to place the tubing into the cassette. In addition, the outer diameter of the tubing is no greater than 3 mm in order to limit the fluid losses. Further, the outer diameter of the tubing in some embodiments is equal to 2.4 mm so that it may be adapted to commercially available connectors.

A pinch valve as described above, i.e. comprising a movable pin and a fixed stop for pinching a flexible tube in order to control a fluid flow rate in the flexible tube, may be described as a “two-state” pinch valve, these two states being an “open” state, where the flexible tube is not pinched and therefore allows a maximum fluid flow rate to pass, and a “closed” state, where the flexible tube is pinched between the pin and the stop and therefore allows a minimum fluid flow rate to pass, or even where the fluid flow rate through the flexible tube is completely interrupted, in which case the minimum flow rate is zero. It is important to note that the two aforementioned states are not the only possible states of such a “two-state” pinch valve, but rather that they delimit a continuity of possible states of the valve, where the flexible tube is more or less pinched between the pin and the stop, and corresponding to a continuity of possible flow rates between the aforementioned maximum flow rate and the minimum flow rate. For example, the use of a pinch valve to control a flow rate of a fluid in a flexible tube as a continuum of possible flow rates between a maximum flow rate and a minimum flow rate has the advantage of being able to control the pressure of the fluid in the flexible tube downstream of the valve.

According to one embodiment, the fluid treatment device may also comprise at least one three-state pinch valve. The three-state pinch valve comprises a movable pin and two fixed stops disposed on both sides of the movable pin. The three-state pinch valve being configured for controlling a flow rate of a fluid into either one of two flexible tubes of the tubing by pinching one of the two flexible tubes against one of the two fixed stops by a movement of the movable pin along a trajectory essentially parallel to the essentially flat surface of the cassette.

The three states of such a “three-state” pinch valve configured for controlling a fluid flow rate in either one of a first flexible tube and a second flexible tube are as follows. In the first state, neither tube is pinched and each tube therefore allows maximum fluid flow rate. In a second state, the first flexible tube is pinched between the pin and the first of the two fixed stops and the fluid flow rate through the first flexible tube is reduced to a minimum fluid flow rate, or even completely interrupted, in which case the minimum fluid flow rate is zero, whereas the second flexible tube is not pinched and therefore allows a maximum fluid flow rate to pass through. In a third state, the second flexible tube is pinched between the pin and the second of the two fixed stops and the fluid flow rate passing through the second flexible tube is reduced to a minimum fluid flow rate, or even completely interrupted, in which case the minimum fluid flow rate is zero, whereas the first flexible tube is not pinched and therefore allows a maximum fluid flow rate to pass through.

Furthermore, the remark made above concerning the continuity of possible states for a “two-state” pinch valve also applies, mutatis mutandis, to a “three-state” pinch valve, i.e. the states are not the only possible states of such a three-state pinch valve but rather delimit a continuity of possible states of the valve, where one of the two flexible tubes is more or less pinched between the pin and one of the two stops, thus allowing a continuity of possible flow rates between the aforementioned maximum flow rate and minimum flow rate. Finally, it should be noted that since such a three-state pinch valve comprises only a single movable pin, it offers the possibility of reducing or even interrupting a fluid flow rate in only one of the two flexible tubes at a time.

The use of a “three-state” pinch valve offers the advantage of being able to increase the possible configurations of a fluidic flow diagram implemented by a device according to the disclosure for a given number of pinch valves and/or a given total length of tubes, or alternatively of being able to achieve a given configuration of a fluidic flow diagram using a reduced number of pinch valves and/or using a reduced total length of tubes, thus allowing to reduce the resources, costs, possible points of failure, and maintenance required for such a device.

In an embodiment, the fluid treatment device may also comprise at least one multi-way pinch valve comprising or consisting of an assembly of several pinch valves. For the purposes of the present disclosure, a “port”, also referred to as a “way”, of a pinch valve is defined as a point of inlet and/or outlet for a fluid into such a pinch valve. Generally speaking, it is possible for a port to correspond to a fluid inlet and outlet point in a pinch valve, in which case it will be referred to as a “bidirectional” port. A pinch valve, as described above, for controlling the flow rate of a fluid through a flexible tube by pinching the flexible tube, comprises two ports and may therefore be described as a “two-way” valve. In such a two-way pinch valve, one of the two ports may be an inlet port and the other an outlet port, or both ports may be bidirectional ports. It is also possible to create a pinch valve with more than two ports, for example by assembling several two-way pinch valves. For example, a so-called “three-way” pinch valve comprising three ports, for example a fluid inlet port and two fluid outlet ports, may comprise two two-way pinch valves, each of these two two-way pinch valves controlling a flow rate of a fluid between the inlet port and one of the two outlet ports of the three-way pinch valve. In this way, a multi-way pinch valve may be produced by assembling several pinch valves, in particular by assembling several two-way pinch valves.

The use of a multi-way pinch valve offers the advantage of being able to increase the possible configurations of a fluidic flow diagram implemented by a device according to the disclosure for a given number of pinch valves and/or a given total length of tubes, or alternatively of being able to achieve a given configuration of a fluidic flow diagram using a reduced number of pinch valves and/or using a reduced total length of tubes, thus allowing to reduce the resources, costs, possible points of failure, and maintenance required for such a device.

In an embodiment, the at least one multi-way pinch valve comprises or consists of a four-way pinch valve, formed by the assembly of four pinch valves. A four-way pinch valve formed by assembling four pinch valves may be produced in a compact manner, thus limiting the overall dimensions of such a four-way pinch valve. In addition, one or more of such four-way pinch valves may be arranged side by side, thus forming a compact and modular valve assembly, allowing a large number of possible configurations of a fluidic flow diagram implemented by a device according to the disclosure to be realized.

In an embodiment, at least one pinch valve comprised in the at least one multi-way pinch valve is a three-state pinch valve, comprising a movable pin and two fixed stops. The use of a three-state pinch valve within a multi-way pinch valve formed by assembling several pinch valves allows to increase the compactness and the modularity of such a multi-way pinch valve, thus allowing a greater number of possible configurations of a fluidic flow diagram implemented by a device according to the disclosure.

In one embodiment, the movable pin of the pinch valve is actuated by a servomotor. For the purposes of the present disclosure, “servomotor” means a position- and/or torque-controlled motor capable of maintaining an opposition to a static force, the position and/or torque of which is continuously checked and corrected as a function of the measurement. One of the advantages of using a servomotor to operate a pinch valve is that the valve may be maintained in several stable positions, unlike prior art pinch valves equipped with a return spring, which may typically only maintain a single stable position if they are not continuously supplied with power, namely the position wherein the return spring is relaxed.

Another advantage of using a servomotor to operate a pinch valve to control a flow rate of fluid through a flexible tube is that it allows a more precise control of the flow rate of fluid through the tube. A pinch valve according to the prior art is generally limited to two limit positions, or states: an “open” state of the valve, where the tube is not pinched and thus allows a maximum flow rate of fluid to pass, and a “closed” state, where the tube is completely pinched and where the flow rate in the tube is thus completely interrupted. On the other hand, when a pinch valve is actuated by a servomotor, as in some embodiments of the present disclosure, the latter may maintain one or more intermediate positions between the two limit positions, thus allowing more precise control of the flow rate of the fluid through the tube.

In one embodiment, at least one flexible tube is made of silicone. One of the advantages of using silicone as a material for a flexible tube is that it is inexpensive and has interesting properties for treating fluids in pharmaceutical and radiopharmaceutical applications requiring a high degree of purity of the fluids treated, such as low reactivity with the fluids circulating through the tube, the ability to be used in a wide range of temperatures, from −40° C. to +200° C. and even higher, and good mechanical strength.

According to another aspect, an apparatus for synthesizing radiopharmaceutical compounds is provided, comprising a tubing that may be easily, quickly, and cost-effectively installed and replaced. In an embodiment, such an apparatus for synthesizing radiopharmaceutical compounds comprises a fluid treatment device according any one of the embodiments disclosed herein.

According to another aspect, a fluid treatment method is provided for allowing a tubing comprised in a fluid treatment device to be easily, quickly, and cost-effectively installed and replaced. In an embodiment, the fluid treatment method comprises: providing a tubing comprising at least one flexible tube that may be pinched (or able to be pinched); providing a cassette having a substantially flat surface for receiving the tubing; assembling the tubing with the cassette; providing a pinch valve comprising a movable pin and a fixed stop, the pinch valve being adapted to pinch the flexible tube for controlling a fluid flow rate in the flexible tube; and mechanically coupling the cassette comprising the pre-assembled tubing with the pinch valve such that the movable pin of the pinch valve describes a trajectory substantially parallel to the substantially flat surface of the cassette when the pinch valve is activated to pinch the flexible tube.

After the step of assembling the cassette with the tubing, the cassette comprises the pre-assembled tubing. In an embodiment of the method, the tubing is first assembled (or pre-assembled) with the cassette before the cassette is mechanically coupled with the pinch valve.

In an embodiment, the method further comprises circulating a fluid through the tubing to treat this fluid.

In an embodiment, the method further comprise replacing the cassette comprising the pre-assembled tubing.

The cassette replacement allows the tubing pre-assembled with the cassette to be replaced quickly and easily. For example, this replacement may be performed without having to dismantle the pinch valve. It may be advantageous to replace the cassette comprising the pre-assembled tubing in order to implement a new fluid treatment scheme corresponding to a new configuration of the tubing. This new fluid treatment scheme may be achieved by means of a new tubing pre-assembled with a new cassette. The cassette may also be replaced in order to limit or reduce a risk of contamination of a fluid circulating in the tubing, such as a contamination of this fluid by a fluid that has previously circulated in the tubing. The cassette may also be replaced if the tubing is worn or damaged.

In an embodiment, the method further comprises: periodically replacing the cassette comprising the pre-assembled tubing.

It may be advantageous to periodically replace the cassette comprising the pre-assembled tubing in order to limit or reduce a risk of contamination of a fluid circulating in the tubing, such as a contamination of this fluid by a fluid which has previously circulated in the tubing. The cassette may also be replaced periodically to replace worn or damaged tubing.

In the case where the fluid treatment device comprises a base comprising the at least one pinch valve, one embodiment of the method may comprise: providing a tubing comprising at least one flexible tube that may be pinched (or able to be pinched); providing a cassette having a substantially flat surface to receive the tubing; assembling the tubing with the cassette; providing a base comprising a pinch valve, the pinch valve comprising a movable pin and a fixed stop, the pinch valve being adapted to pinch the flexible tube for controlling a fluid flow rate in the flexible tube; and then mechanically coupling the cassette comprising the pre-assembled tubing with the base such that the movable pin of the pinch valve describes a trajectory substantially parallel to the substantially flat surface of the cassette when the pinch valve is activated to pinch the flexible tube.

### DESCRIPTION OF THE DRAWINGS

Other characteristics and advantages of the present disclosure will become apparent from the following detailed description, for the understanding of which reference is made to the attached figures, among which:

FIGS. 1a, 1b, 1c, 1d schematically illustrate a two-state pinch valve according to an embodiment of the disclosure;

FIGS. 2a, 2b, 2c schematically illustrate a three-state pinch valve according to an embodiment of the disclosure;

FIG. 3a schematically illustrates a multi-way pinch valve according to an embodiment of the disclosure and formed by the assembly of several pinch valves;

FIG. 3b schematically illustrates a fluid treatment device according to an embodiment of the disclosure and comprising such a multi-way pinch valve;

FIGS. 4a, 4b are a schematic representation of a multi-way pinch valve according to an embodiment of the disclosure and actuated by servomotors;

FIG. 5 is a schematic representation of a fluid treatment device comprising a cassette comprising a pre-assembled tubing, and a plurality of multi-way pinch valves; and

FIG. 6 is a schematic representation of an apparatus for synthesizing radiopharmaceutical compounds comprising a fluid treatment device.

The drawings in the figures are not to scale. Generally, similar elements are denoted by similar references in the figures. For the purposes of this document, identical or similar items may bear the same references. Furthermore, the presence of reference numbers in the drawings may not be regarded as limiting, even when these numbers are indicated in the claims.

### DETAILED DESCRIPTION

The present disclosure will be described with respect to particular embodiments and with reference to certain drawings but the disclosure is not limited thereto. The drawings or figures described herein are only schematic and are non-limiting. In addition, the functions described in this document may be carried out by other structures than those described or illustrated in this document.

Use of the verb “to comprise”, as well as the respective variants and conjugations, does not exclude the presence of elements other than those stated. Use of the article “a”, “an” or “the” preceding an element does not exclude the presence of a plurality of such elements.

The terms “first”, “second”, “third” and the like, in the description and in the claims, are used for distinguishing between similar elements and not necessarily for describing a sequential or chronological order. The terms are interchangeable under appropriate circumstances and the embodiments of the disclosure can operate in other sequences than those described or illustrated herein.

Furthermore, embodiments referred to as “preferred” are to be construed as exemplary manners in which the disclosure may be implemented rather than as limiting the scope of the disclosure.

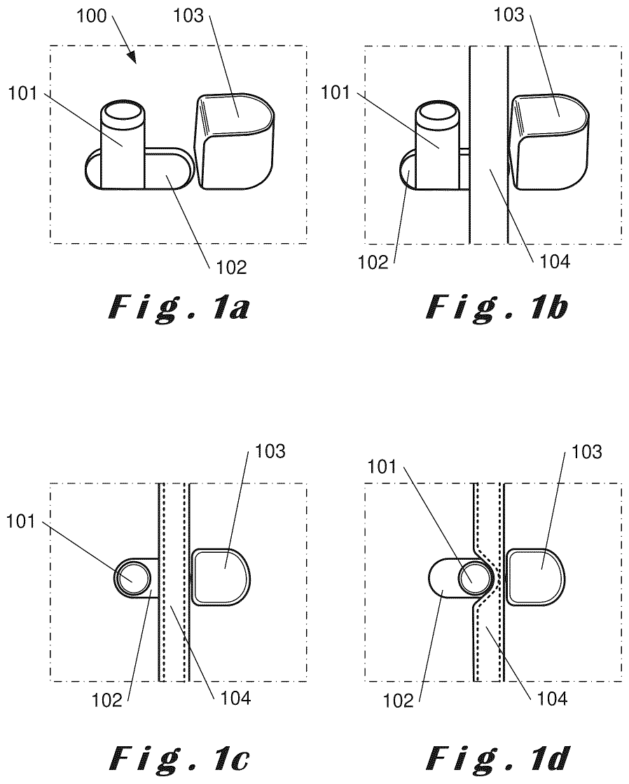

FIGS. 1a, 1b, 1c, 1d schematically illustrate a two-state pinch valve 100 according to one embodiment of the disclosure. As shown in FIG. 1a, the two-state pinch valve 100 comprises a movable pin 101 which may move in a notch 102, and a fixed stop 103. As shown in FIG. 1b, a flexible tube 104 may be placed between the pin 101 and the stop 103 with a view to being pinched against the stop 103 by a movement of the pin 101 in the notch 102 in order to control a flow rate of a fluid in the tube 104. FIG. 1c illustrates an “open” state of the two-state pinch valve 100, where the flexible tube 104 is not pinched and therefore allows a maximum fluid flow rate. FIG. 1d illustrates a “closed” state of the two-state pinch valve 100, where the flexible tube 104 is pinched between the pin 101 and the stop 103 and where the flow rate of fluid through the flexible tube 104 is completely interrupted.

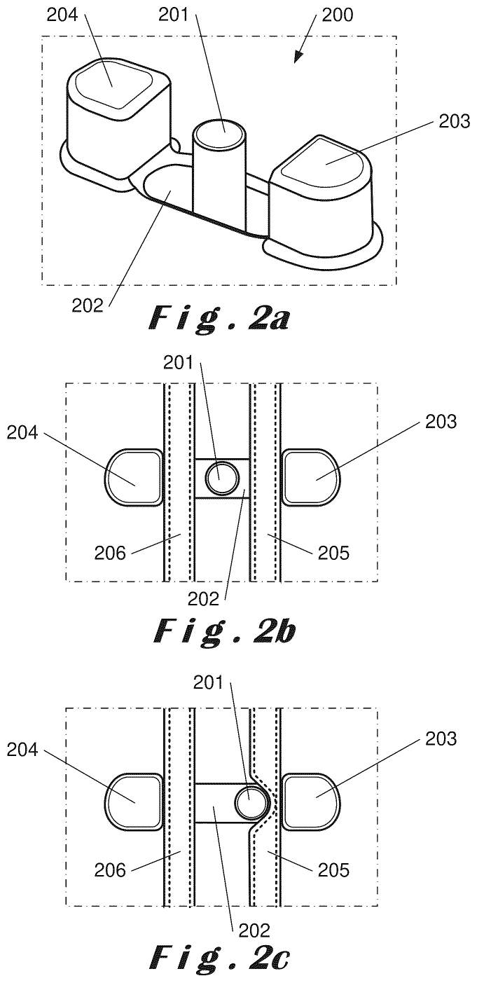

FIGS. 2a, 2b, 2c schematically illustrate a three-state pinch valve 200 according to one embodiment of the disclosure. As shown in FIG. 2a, the three-state pinch valve 200 comprises a movable pin 201 which may move in a notch 202, and two fixed stops 203, 204 arranged on either side of the pin 201. As shown in FIGS. 2b and 2c, two flexible tubes 205, 206 may be placed between the pin 201 and each of the two stops 203, 204 so that the pin 201 may pinch one of the two flexible tubes 205, 206 against one of the two stops 203, 204 to control a flow rate of fluid in this tube. FIG. 2b illustrates a first state of the three-state pinch valve 200, where neither of the two tubes 205, 206 is pinched and where each of the two tubes 205, 206 therefore allows a maximum fluid flow rate. FIG. 2c illustrates a second state of the three-state pinch valve 200, where the tube 205 is pinched between the pin 201 and the stop 203 and the fluid flow rate through the tube 205 is completely interrupted, while the tube 206 is not pinched and therefore allows maximum fluid flow rate.

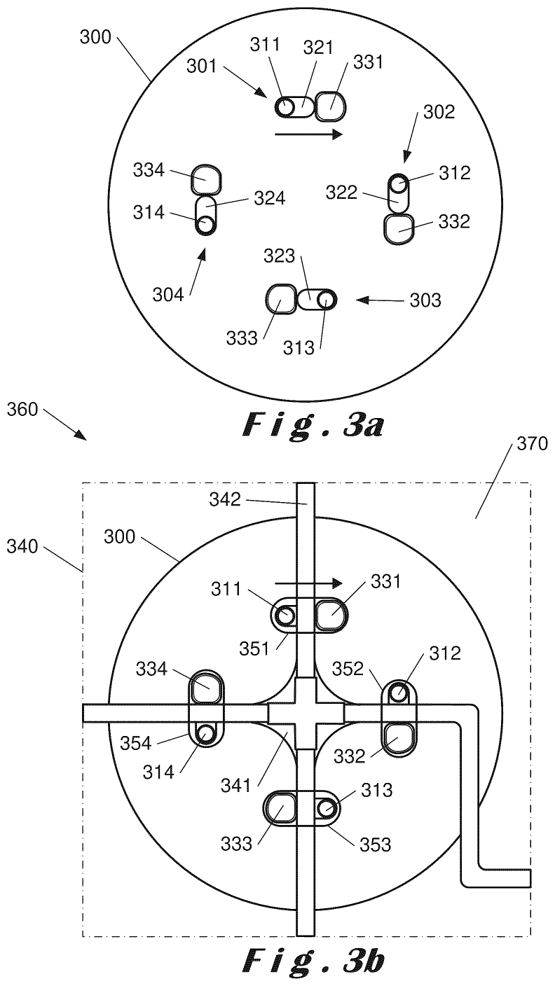

FIG. 3a schematically illustrates a multi-way pinch valve 300 according to one embodiment of the disclosure and formed by the assembly of several pinch valves 301, 302, 303, 304, while FIG. 3b schematically illustrates a fluid treatment device 360 according to one embodiment of the disclosure and comprising the multi-way pinch valve 300. For example, FIG. 3a illustrates a four-way pinch valve 300 formed by the assembly of four pinch valves 301, 302, 303, 304, each of these four pinch valves 301, 302, 303, 304 comprising a movable pin 311, 312, 313, 314 movable in a notch 321, 322, 323, 324, as well as a fixed stop 331, 332, 333, 334. The arrow next to the pinch valve 301 illustrates the direction along which the pin 311 may move in the notch 321 towards the stop 331. FIG. 3b illustrates a device 360 for fluid treatment comprising the four-way pinch valve 300 and a cassette 340 comprising a network of support channels 341 comprising a tubing 342. The cassette 340 is essentially made of a thermoplastic polymer. The cassette 340 is also transparent in some embodiments. The cassette 340 has an essentially flat surface 370 to receive the tubing 342. The network of support channels 341 further comprises clearance slots 351, 352, 353, 354 to receive the movable pin 311, 312, 313, 314 and the fixed stop 331, 332, 333, 334 comprised in each of the pinch valves 301, 302, 303, 304. Each of the clearance slots 351, 352, 353, 354 is in the form of an oval hole cut into the thickness of the cassette 310. These clearance slots 351, 352, 353, 354 facilitate the coupling between the tubing 342 comprised in the cassette 340 and the pinch valves 301, 302, 303, 304. Finally, as shown in FIG. 3a, an arrow illustrates the direction along which the pin 311 may move towards the stop 331 to pinch a flexible tube of the tubing 342 by describing a trajectory essentially parallel to the essentially flat surface 370 of the cassette 340.

The multi-way pinch valve 300 is an example of a base 300 that may be mechanically coupled with a cassette 340 comprising a pre-assembled tubing 342.

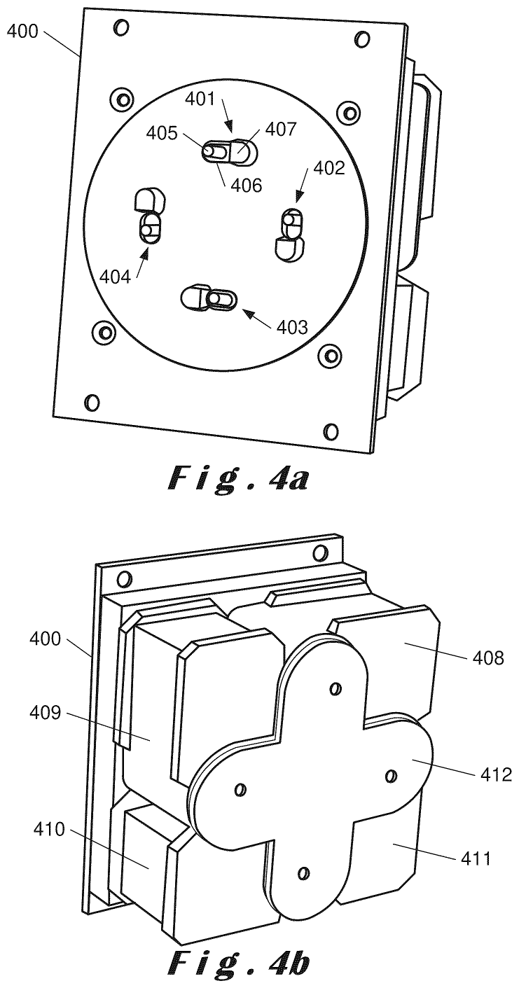

FIGS. 4a and 4b are a schematic representation of a multi-way pinch valve 400 according to one embodiment of the disclosure and actuated by servomotors. For example, FIGS. 4a and 4b are a perspective view of the four-way pinch valve 300 shown in FIG. 3a, in a case where this pinch valve 300 is actuated by servomotors. The pinch valve 400 is therefore also an example of a base that may be mechanically coupled with a cassette 340 comprising a pre-assembled tubing 342.

FIG. 4a shows a front view of the four-way pinch valve 400, while FIG. 4b shows a rear view of the four-way pinch valve 400. The pinch valve 400 comprises four pinch valves 401, 402, 403, 404, corresponding respectively to the pinch valves 301, 302, 303, 304 shown in FIGS. 3a and 3b. Each of the pinch valves 401, 402, 403, 404 comprises a movable pin which may move in a notch, as well as a fixed stop, such as the movable pin 405, the notch 406, and the fixed stop 407 for the pinch valve 401. The pinch valves 401, 402, 403, 404 are respectively actuated by servomotors 408, 409, 410, 411, these servomotors being held by a rear support 412.

A multi-way pinch valve 400 as shown in FIGS. 4a and 4b has a number of advantages. Firstly, a pinch valve 400 of this type has a small overall dimension. The pinch valves 401, 402, 403, 404 comprised in the pinch valve 400 may be placed relatively close to each other, allowing to create a compact multi-way pinch valve.

In addition, the pinch valves 401, 402, 403, 404 are located in the same plane, which means that the tubing comprised in a cassette may be coupled to the assembly of the pinch valves in a single operation, as described above. The multi-way pinch valve 400 is an example of a base comprising a plurality of pinch valves 401, 402, 403, 404 and with which a cassette 340 comprising a pre-assembled tubing 342 and having a substantially flat surface 370 for receiving the tubing 342 may be mechanically coupled. More precisely, the cassette 340 may be coupled with this base so that the movable pin of each of the pinch valves 401, 402, 403, 404 comprised in the base describes a trajectory substantially parallel to the substantially flat surface 370 of the cassette 340 when the pinch valves 401, 402, 403, 404 is activated to pinch a flexible tube of the tubing 342. Preferably, the cassette 340 is removably coupled to this base.

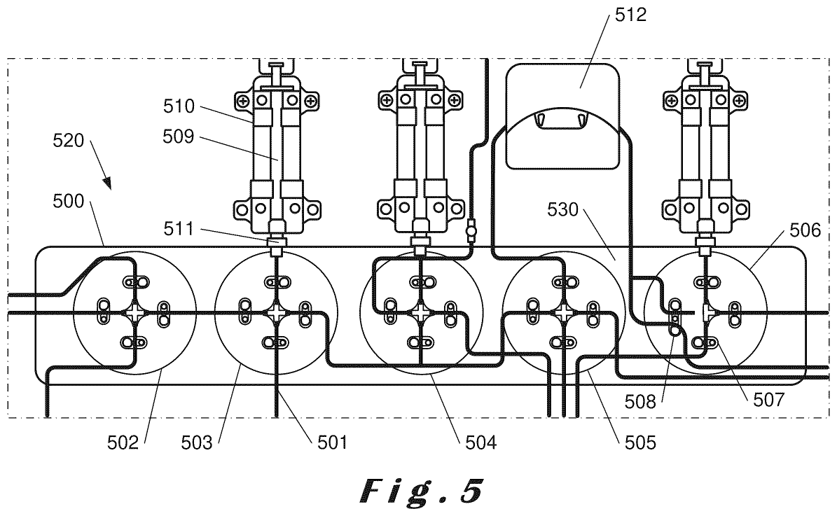

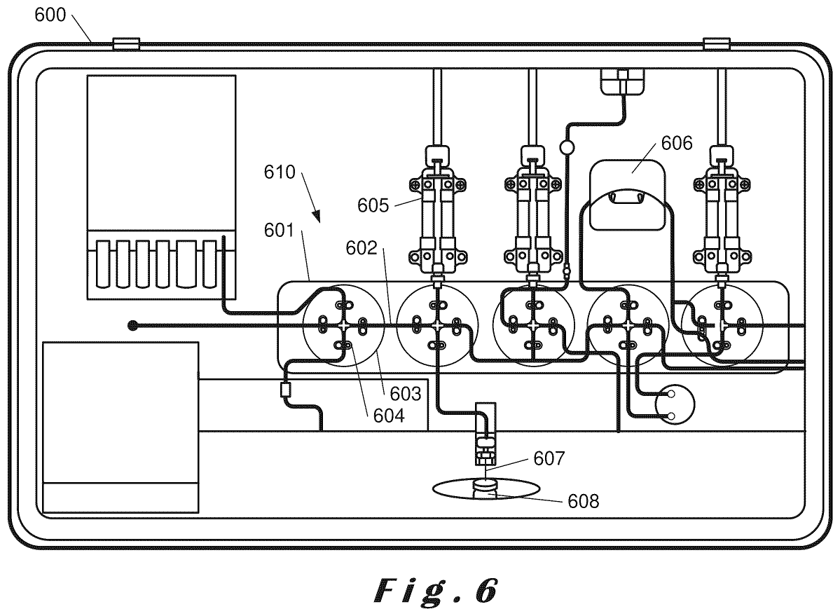

Further, as the actuators (servomotors) 408, 409, 410, 411 are compactly arranged at the rear of the pinch valve 400, it is possible to arrange several examples of this pinch valve 400 side by side to form a compact and modular valve assembly, as illustrated in FIG. 5 and FIG. 6.

FIG. 5 is a schematic representation of a fluid treatment device 520 according to one embodiment of the disclosure. The fluid treatment device 520 comprises a cassette 500 comprising a tubing 501 pre-assembled and mechanically coupled to a plurality of multi-way pinch valves 502, 503, 504, 505, 506. The cassette 500 has an essentially flat surface 530 for receiving the tubing 501. The multi-way pinch valves 502, 503, 504, 505, 506 form a base with which the cassette 500 comprising the pre-assembled tubing 501 may be mechanically coupled. Each of the multi-way pinch valves 502, 503, 504, 505, 506 may comprise one or more two-state pinch valves 507 and/or one or more three-state pinch valves 508. In addition, the tubing 501 comprised in the cassette 500 may be coupled to one or more devices capable of handling a fluid or measuring a physico-chemical property of the fluid, such as for example: a syringe 509 which may be actuated by a syringe pump 510 and coupled to the tubing 501 by means of a connector such as a “Luer” connector 511; or a peristaltic pump 512.

FIG. 6 is a schematic representation of an example of an apparatus 600 for synthesizing radiopharmaceutical compounds comprising a fluid treatment device 610 according to one or more embodiments of the disclosure. The fluid treatment device 610 of FIG. 6 corresponds to the fluid treatment device 520 in FIG. 5. The device 610 comprises a cassette 601 comprising a tubing 602 mechanically coupled to a plurality of multi-way pinch valves 603, each of which multi-way pinch valves 603 may comprise one or more pinch valves 604. The plurality of multi-way pinch valves 603 is an example of a base with which the cassette 601 comprising the pre-assembled tubing 602 may be mechanically coupled. In addition, the tubing 602 comprised in the cassette 601 may be coupled to one or more devices capable of handling a fluid or measuring a physico-chemical property of the fluid, such as: a syringe that may be actuated by a syringe pump 605; a peristaltic pump 606; a needle 607 for injecting or taking a fluid sample in a flask 608.

In summary, the disclosure relates to a fluid treatment device 360, 520 comprising: a cassette 340, 500 comprising a pre-assembled tubing 342, 501, the cassette 340, 500 having a substantially flat surface 370, 530 for receiving the tubing 342, 501, the tubing 342, 501 comprising at least one flexible tube 104 able to be pinched; at least one pinch valve 100, 200 comprising a movable pin 101 and a fixed stop 103 for pinching the flexible tube 104 in order to control a fluid flow rate in the flexible tube; wherein the at least one pinch valve 100, 200 is mechanically coupled to the cassette 340, 500 such that the movable pin 101 of the at least one pinch valve 100, 200 describes a trajectory substantially parallel to the substantially flat surface 370, 530 of the cassette when the at least one pinch valve 100, 200 is activated to pinch the flexible tube 104. The tubing 342, 501 comprised in the fluid treatment device 360, 520 according to the disclosure may be easily, quickly, and cost-effectively installed and replaced. The disclosure also relates to a fluid treatment method allowing a tubing 342, 501 comprised in a fluid treatment device 360, 520 according to the disclosure to be easily, quickly and cost-effectively installed and replaced.

In the foregoing description, specific details are set forth to provide a thorough understanding of representative embodiments of the present disclosure. It will be apparent to one skilled in the art, however, that the embodiments disclosed herein may be practiced without embodying all of the specific details. In some instances, well-known process steps have not been described in detail in order not to unnecessarily obscure various aspects of the present disclosure.

Although the method and various embodiments thereof have been described as performing sequential steps, the claimed subject matter is not intended to be so limited. As nonlimiting examples, the described steps need not be performed in the described sequence and/or not all steps are required to perform the method. Moreover, embodiments are contemplated in which various steps are performed in parallel, in series, and/or a combination thereof. As such, one of ordinary skill will appreciate that such examples are within the scope of the claimed embodiments.

In the detailed description herein, references to “one embodiment”, “an embodiment”, “an example embodiment”, “one or more embodiments”, “some embodiments”, etc., indicate that the embodiment or embodiments described may include a particular feature, structure, or characteristic, but every embodiment may not necessarily include the particular feature, structure, or characteristic. Moreover, such phrases are not necessarily referring to the same embodiment or embodiments. In addition, when a particular feature, structure, or characteristic is described in connection with an embodiment or embodiments, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments. Thus, it will be appreciated that embodiments of the present disclosure may employ any combination of features described herein. All such combinations or sub-combinations of features are within the scope of the present disclosure.

Throughout this specification, terms of art may be used. These terms are to take on their ordinary meaning in the art from which they come, unless specifically defined herein or the context of their use would clearly suggest otherwise.

The present application may reference quantities and numbers. Unless specifically stated, such quantities and numbers are not to be considered restrictive, but exemplary of the possible quantities or numbers associated with the present application. Also in this regard, the present application may use the term “plurality” to reference a quantity or number. In this regard, the term “plurality” is meant to be any number that is more than one, for example, two, three, four, five, etc. The terms “about,” “approximately,” “near,” etc., mean plus or minus 5% of the stated value. For the purposes of the present disclosure, the phrase “at least one of A and B” is equivalent to “A and/or B” or vice versa, namely “A” alone, “B” alone or “A and B.”. Similarly, the phrase “at least one of A, B, and C,” for example, means (A), (B), (C), (A and B), (A and C), (B and C), or (A, B, and C), including all further possible permutations when greater than three elements are listed.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. While the stated range includes one or both of the limits, ranges excluding either or both of those included limits are also included in the disclosure

The principles, representative embodiments, and modes of operation of the present disclosure have been described in the foregoing description. However, aspects of the present disclosure which are intended to be protected are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure, as claimed.

1. A fluid treatment device comprising:
a cassette comprising a pre-assembled tubing, the cassette having a substantially flat surface for receiving the tubing, the tubing comprising at least one flexible tube able to be pinched;
at least one pinch valve comprising a movable pin and a fixed stop for pinching the flexible tube in order to control a fluid flow rate in said flexible tube;
wherein the at least one pinch valve is mechanically coupled to the cassette such that the movable pin of the at least one pinch valve describes a trajectory substantially parallel to the substantially flat surface of the cassette when the at least one pinch valve is activated to pinch the flexible tube.

2. The fluid treatment device according to claim 1, further comprising a base comprising the at least one pinch valve, wherein the cassette is mechanically coupled with the base such that the movable pin of the at least one pinch valve describes a trajectory essentially parallel to the essentially flat surface of the cassette when the at least one pinch valve is activated to pinch the flexible tube.

3. The fluid treatment device according to claim 2, wherein the cassette is coupled to the base removably and independently of the at least one pinch valve.

4. The fluid treatment device according to claim 1, wherein the cassette further comprises a network of support channels for accommodating the pre-assembled tubing, said network of support channels preferably comprising, for each pinch valve, a clearance slot allowing the movable pin and the fixed stop to be mounted around the flexible tube.

5. The fluid treatment device according to claim 1, wherein the cassette is partially made of a thermoplastic polymer.

6. The fluid treatment device according to claim 1, wherein the pre-assembled tubing comprises at least one connector allowing the tubing to be coupled to at least one device capable of manipulating the fluid or measuring a physico-chemical property of the fluid.

7. The fluid treatment device according to claim 1, wherein the tubing includes a network of cylindrical tubes with an outer diameter of between 1 mm and 10 mm.

8. The fluid treatment device according to claim 1, further comprising at least one three-state pinch valve, such a three-state pinch valve comprising a movable pin and two fixed stops disposed on both sides of the movable pin, said three-state pinch valve being configured for controlling a flow rate of a fluid into either one of two flexible tubes of the tubing by pinching one of the two flexible tubes against one of the two fixed stops by a movement of the movable pin along a trajectory essentially parallel to the essentially flat surface of the cassette.

9. The fluid treatment device according to claim 1, further comprising at least one multi-way pinch valve including an assembly of several pinch valves.

10. The fluid treatment device according to claim 9, wherein the at least one multi-way pinch valve includes a four-way pinch valve, formed by the assembly of four pinch valves.

11. The fluid treatment device according to claim 9, wherein at least one pinch valve comprised in the at least one multi-way pinch valve is a three-state pinch valve, comprising a movable pin and two fixed stops.

12. The fluid treatment device according to claim 1, wherein the movable pin of the pinch valve is actuated by a servomotor.

13. An apparatus for synthesizing radiopharmaceutical compounds comprising a fluid treatment device according to claim 1.

14. The fluid treatment device according to claim 7, wherein the outer diameter is between 2 mm and 3 mm.

15. The fluid treatment device according to claim 14, wherein the outer diameter is equal to 2.4 mm.

16. A fluid treatment method comprising:
providing a tubing comprising at least one flexible tube able to be pinched;
providing a cassette having a substantially flat surface for receiving the tubing;
assembling the tubing with the cassette;
providing a pinch valve comprising a movable pin and a fixed stop, the pinch valve being adapted to pinch the flexible tube for controlling a fluid flow rate in said flexible tube; and thereafter
mechanically coupling the cassette with the pinch valve such that the movable pin of the pinch valve describes a trajectory substantially parallel to the substantially flat surface of the cassette when the pinch valve is activated to pinch the flexible tube.

17. The fluid treatment method according to claim 16, further comprising:
replacing the cassette comprising the pre-assembled tubing.
